(19) United States
(12) Patent Application Publication
(10) Pub. No.: US 2006/0240413 A1
(43) Pub. Date: Oct. 26, 2006

(54) Medical test probe for cell sample collection

(75) Inventor: Sandra K. Auttebery, Westcliffe, CO (US)

Correspondence Address: WESTMAN CHAMPLIN & KELLY, P.A., SUITE 1400, 900 SECOND AVENUE SOUTH, MINNEAPOLIS, MN, 55402-3319, US

(73) Assignee: Medical Diagnostic Innovations, LLC, Canon City, CO (US)

(21) Appl. No.: 11/408,655

(22) Filed: Apr. 21, 2006

Related U.S. Application Data
(60) Provisional application No. 60/673,410, filed on Apr. 21, 2005.

(51) Int. Cl. C12Q 1/70; C12Q 1/04; C12M 1/34; A61M 31/00

(52) U.S. Cl. 435/5; 435/34; 435/287.1; 604/95.01

(57) ABSTRACT

A device and method are disclosed for collecting biological samples. One embodiment includes a device for collecting biological samples comprising a probe having a cell collector located on a distal end, the probe having a plurality of distinct raised protrusions.

The present application is based on and claims the benefit of U.S. provisional patent application Ser. No. 60/673,410, filed Apr. 21, 2005, the content of which is hereby incorporated by reference in its entirety.

### BACKGROUND OF THE INVENTION

The present invention generally pertains to medical testing devices. In particular, the present invention pertains to a medical test probe for cell sample collection, wherein the probe represents a component used within a female at-home testing kit.

Medical testing procedures are an important aspect of an individuals personal health and well being. Because it is important to identify the cause of illness and diseases at an early stage, some medical testing procedures are designed as precautionary measures that are performed on a regularly scheduled basis (i.e., annually). On the other hand, some medical testing procedures are performed after the onset of symptoms such as pain, infections, rashes, irritations, etc. In many instances, early detection results in an increased change of cure or a reduction of damage done to the body.

Despite the advantages of identifying illness and disease at an early stage, many people do not have regular medical examination procedures performed as recommended by medical personnel (i.e., physicians, nurses, etc.). Further, even after the onset of physical symptoms many people still refuse to are cannot have appropriate medical examination performed. For example, many women do not have regular pap smear screening tests.

There are a number of reasons why people do not have medical procedures (e.g., such as pap smears) performed on a regularly recommended basis. For example, some people are unable to afford the usual $200.00, or greater, fee it costs for a medical screening test in a physician's office. Further, some people have had negative experiences in their lives such as rape, incest and/or are just so private or shy that they avoid the life saving tests. Further yet, some people live in remote areas, nationally and internationally, where traveling to reach medical care is a hardship so therefore they do not have the exam performed regularly.

Statistics reveal that a great many number of people die yearly because of failure to have medical testing performed for reasons such as those mentioned above. As such, there exists a need to provide adequate medical testing procedures that encourage individuals to receive regular exams such as pap smear screening tests.

### SUMMARY OF THE INVENTION

Embodiments of a device and system are disclosed for collecting biological samples. The embodiments include a device for collecting biological samples from a cavity accessed through a vaginal orifice. The device includes a probe having a cell collector located on a distal end, the probe having a plurality of distinct raised protrusions.

Another embodiment includes a system for collecting biological samples comprising a probe having a proximal end and a distal end, a cell collector located on the distal end of the probe, and a preservation package configured to at least partially enclose the cell collector.

Another embodiment includes a method for collecting a biological sample from a cavity accessed through a vaginal orifice. The method comprises inserting a cell collection probe into the orifice, rotating the cell collection probe, removing the cell collection probe from the orifice, and placing the cell collection probe in a preservation tube.

### BRIEF DESCRIPTION OF THE DRAWINGS

FIG. 1 is a side view of a medical testing device.

FIG. 2 is a side cross-sectional view of a medical testing device.

FIG. 3A is a side view of the medical testing probe of FIG. 2.

FIG. 3B is a perspective view of the medical testing probe of FIG. 2.

FIG. 3C is an end view of the medical testing probe of FIG. 2.

FIG. 4 is a side cross-sectional view of a portion of a medical testing device.

FIGS. 5A-5D are illustrative view of a medical testing probe.

### DETAILED DESCRIPTION OF ILLUSTRATED EMBODIMENTS

In accordance with one aspect of the present invention, a medical device is provided for medical testing that will enable the end-user to receive a test result such as, but not limited to, a negative or a positive test reading. The device can be utilized as a testing component for any of a variety of medical tests such as, but not limited to, pap smear tests or tests for venereal diseases.

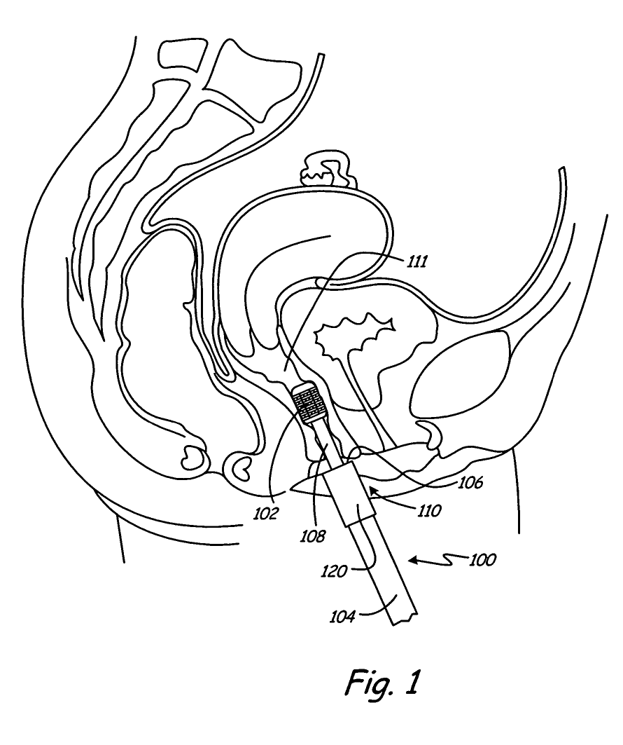

FIG. 1 illustrates a medical testing device 100 in accordance with aspects of the present invention. Device 100 comprises a probe and a guide mechanism. The probe illustratively includes a cell collector 102 and a handle 108. Further, the guide mechanism is configured to guide insertion of the probe into an anatomical orifice, or opening, 110 (e.g., a vagina). The guide mechanism illustratively includes a tube 104 and a tip portion 120.

As is illustrated in FIG. 1, device 100 can be inserted into a female vagina for purposes of conducting a pap smear test, sexual transmitted disease (STD) test, or any other form of medical test or evaluation. It is important to note that other uses of device 100 are within the scope of the present invention.

In accordance with one embodiment, device 100 is inserted at least partially into orifice 110. In other embodiments, the insertion depth of device 100 into orifice 110 can be greater than or less than that illustrated in FIG. 1. For example, tube 104 can be inserted such that end 106 is proximate orifice 110. In another example, device 100 can be inserted approximately an inch into orifice 110. It is noted that FIG. 1 simply illustrates a method of inserting a cell collection probe and is not intended to be an exact diagram of a female anatomy. FIG. 1 may differ slightly from an actual human anatomy.

After device 100 has been inserted at least partial into vaginal opening 110, handle 108 is utilized to further extend cell collector 102 to collect a biological (e.g., cell) sample. Cell collector 102 is extended until it meets resistance from cavity 111 (e.g., a cervix). In one embodiment, cell collector 102 is inserted until it meets the opening of the cervix. In this manner, cell collector 102 is not inserted into the cervix. Cell collector 102 is then rotated to encourage a greater collection of cell samples from the areas surrounding the cervix opening. It is again noted that FIG. 1 simply illustrates a method of insertion and is not intended to illustrate any specific dimensions or depths.

Once cell collector 102 has been utilized and a sample has been collected, cell collector 102 is retracted into tube 104 and tube 104 is removed from orifice 110. In accordance with one embodiment, after device 100 has been utilized to collect cells and removed, cell collector 102 is sealed in a container and sent by mail to a lab for analysis. In accordance with another embodiment, a container for sealing and sending cell collector 102 to a lab is sold and/or furnished with device 100. Subsequently, the lab can communicate results of the tests (e.g., mailed or emailed), to the end-user (i.e., the individual who utilized the probe). If the test results are positive, the end-user is illustratively advised to seek a doctor.

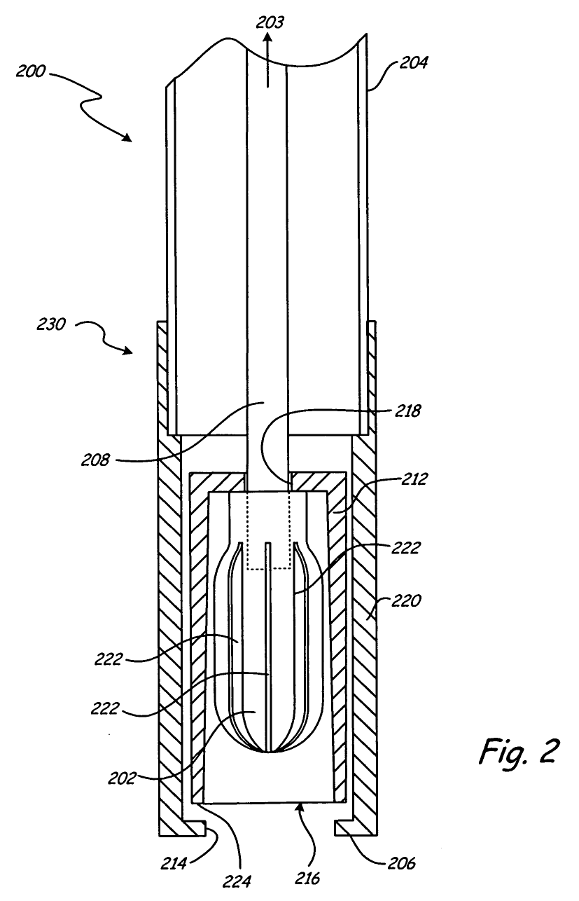

FIG. 2 illustrates a cross-sectional view of a medical testing device 200 in accordance with aspects of the present invention. Device 200 is substantially similar to device 100 illustrated in FIG. 1. Device 200 includes a cell collector 202 configured to be utilized to collect cells. Device 200 further includes a tube 204, a tip portion 220, and a cup portion 212. While the structure and use of cell collector 202 will be describe hereafter in conjunction with tube 204, tip 220, and cup 212, it is important to note that cell collector 202 can be utilized as a stand-alone testing device. In other words, cell collector 202 can be utilized without a guide device.

In accordance with one embodiment, tip 220 is fixedly attached to tube 204. Tip 220 and tube 204 together comprise tip/tube assembly 230. Tip 220 and tube 204 can be constructed of the same or different materials. Further, tip/tube assembly 230 can alternatively be constructed as one-piece. Suitable materials for tip 220 and tube 204 include, but are not limited to, plastic and cardboard.

In accordance with one embodiment, assembly 230 is utilized to guide cell collector 202 into the human anatomy for purposes of cell retrieval. Further, in one embodiment, tube 204 is approximately 0.51 inches in diameter and approximately 4.6 inches in length. In another embodiment, tip/tube assembly 230 is approximately six inches in length. However, any other suitable dimensions of tube 204 are within the scope of the present invention. For example, the diameter of tube 204 can be greater than or less than 0.51 inches to accommodate different probe sizes.

In accordance with one embodiment, cell collector 202 is housed within cup 212. Cup 212 is configured to not protrude beyond tip 220. In one embodiment, cup 212 is constructed of a plastic. In another embodiments, cup 212 can be a polypropylene or polyethylene base. However, cup 212 can be constructed of any suitable material. Cup 212 and cell collector 202 are movable within tip/tube assembly 230.

In one embodiment, an opening 214 is provided in tip 220. Opening 214 in the proximal end 206 of tip 220 is configured to be larger than cell collector 202 but smaller than end 224 of cup 212. As such, cup 212 is discouraged from extending beyond the end 206 of tip 220. Cup 212 can illustratively be removed in a direction indicated by arrow 203 through tube 204 (e.g., when cell collector 202 is retracted, it engages cup 212 and pulls it out of tube 204). In other embodiments, the proximal end 206 of tip 220 can be closed (i.e., it can have a rounded star-shaped closure that can be pushed open by cell collector 202).

Cell collector 202 is attached to handle 208 which can be utilized to extend cell collector 202 through openings 216 and 214 for cell retrieval purposes. In one embodiment, cell collector 202 is manufactured separate from handle 208 and attached thereto. In another embodiment, cell collector 202 and handle 208 are constructed as one piece.

Cell collector 202 illustratively includes scraping mechanisms (e.g., protrusions 222) that accommodate sample collection when the probe is spun, rotated, or turned. However, any configuration of cell collector 202 is within the scope of the present invention.

Further, cell collector 202 can be constructed of any material suitable for gathering cells. Suitable materials can be, but are not limited to, plastic, nylon, and cotton. Further, it is important to note that other configurations of cell collector 202 are within the scope of the present invention. For example, cell collector 202 can be smooth, or include grooves instead of protrusions or fins.

In accordance with one embodiment, cell collector 202 is configured to slide through an opening in tip 220 and exit therefrom in order to facilitate sample collection. Then, the probe is retracted into cup 212 to capture the sample (e.g., cells). Cell collector 202 and cup 212 are configured to be removed from the tip/tube assembly 230 and placed in a transport container (not shown).

One embodiment of the present invention pertains to a procedure for using device 200. First, the test probe assembly 200 is inserted into a vaginal cavity (as shown in FIG. 1) until it meets resistance from the cavity (the tip 220 and tube 204 are inserted until resistance is encountered). Next, pressure is applied on the proximal end of the probe (i.e., the proximal end of handle 208) until resistance is encountered. In one example, cell collector 202 is inserted until it meets the opening of the cervix. Next, cell collector 202 is turned (e.g., handle 208 is rotated clockwise three revolutions). The user then pulls back on the proximal end of handle 208 thereby moving cell collector 202 into cup 212. The entire assembly 200 is then withdrawn out of the vaginal cavity. It is noted that different depths of insertion are within the scope of the present invention.

After removal of device 200 from the cavity, the user pulls on the proximal end of handle 208 (in a direction indicated by arrow 203) so as to pull cell collector 202 out of the proximal end of tube 204. Because cell collector 202 is larger in size than aperture 218 in cup 212, cup 212 is pulled through tube 204 along with cell collector 202. After removal from assembly 200, the user illustratively avoids touching cell collector 202 as to avoid contamination of the cell sample. Handle 208, cup 212, and cell collector 202 can then be placed inside a transport container containing a preserving solution. Alternatively, in some embodiments, handle 208 can be removed such that only cup 212 and cell collector 202 are placed in the transport container. The user illustratively avoids spilling the solution. Finally, the user seals the transport container. The user fills out paperwork and mails the tube to a laboratory address on the mailer. Test results are forwarded to the user.

In one embodiment, the transport container includes a mailing tube and a screw-on cap. In this manner, cell collector 202 can be placed within the transport tube and sealed with the screw-on cap. The transport container can be made out of any number of suitable materials. Some materials that the transport container can be made out of include, but are not limited to, Styrofoam, plastic, cardboard, or metal. In one embodiment, Styrofoam is utilized to provide adequate protection of the contents of the transport container (e.g., to ensure that the contents are not spilled or damaged). However, any suitable configuration of the transport tube is within the scope of the present invention.

In accordance with one embodiment, the transport container includes a preservation solution to enhance the viability of cell samples contained on cell collector 202. The preservation solution can be any number of suitable solutions. In one embodiment, the preservation solution is a methanol-based solution. Methanol-based solutions are widely used, are particularly user-friendly, and have a relatively long shelf life. In another embodiment, less than 10 mL of a methanol-based solution is utilized as a preservation solution. However, any type and amount of preservation solution is within the scope of the present invention.

In accordance with another embodiment, a medical kit is made available for sale that includes a transport container with solution, an assembled medical device including medical probe, instructions for the end-user of the kit regarding use of the medical kit, and laboratory paperwork to be filled out by the end-user. The end-user utilizes the medical probe to collect cells. The end-user then places the medical probe into the transport container and sends the container to a laboratory for testing purposes.

In one embodiment, the end-user returns the laboratory paperwork with the transport container. The laboratory paperwork can include information and questions pertaining to, for example, means for receiving testing results (e.g. the end-user's postal mailing address or email address) and types of tests that are to be performed. For example, the paperwork can include a checklist of illnesses and diseases such as, but not limited to, sexually transmitted diseases(STDs), cancer (e.g., cervical cancer), or the human papilloma virus(HPV). In addition, the list of tests can include a price corresponding to the cost of performing each type of test. Upon receipt by a testing facility, the paperwork can be utilized to determine which tests the end-user has chosen to have performed on the cell sample.

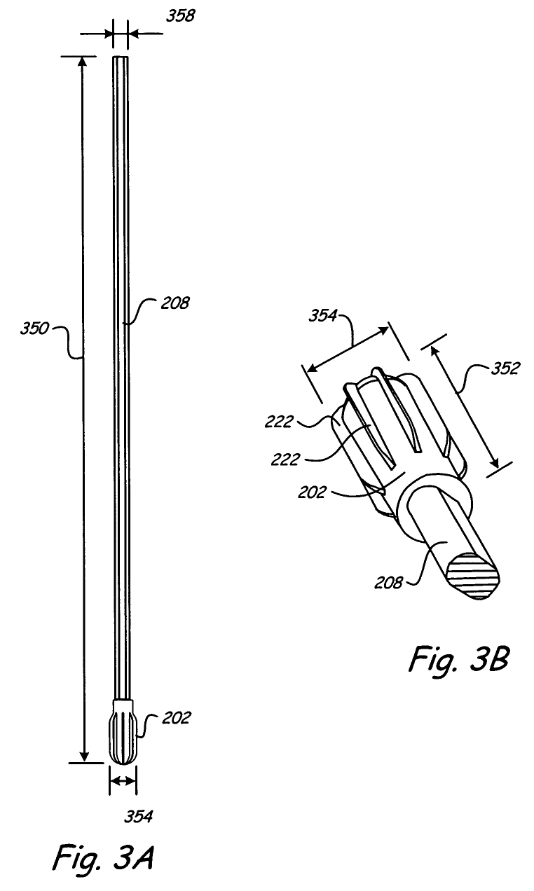

FIGS. 3A, 3B, and 3C are a side view, a perspective view, and an end view, respectively, of the medical testing probe of FIG. 2. In one embodiment, handle 208 has a width 358 of approximately 0.15 inches, a height 360 of approximately 0.1 inches, and a length of approximately seven inches. Handle 208 can be a flexible elongated member manufactured separately and attached to cell collector 202. Alternatively, handle 208 and cell collector 202 can be constructed as one piece. In one embodiment, length 350 from the end of handle 208 to the end of cell collector 202 is 7.3 inches in length. In another embodiment, handle 208 is greater than 7.3 inches in length. In another embodiment, handle 208 is less than 7.3 inches in length. Handle 208 can be constructed of plastic or any other suitable material.

Cell collector 202 also includes scraping mechanisms 222 configured to encourage cell collection. In one embodiment, scraping mechanisms 222 comprise a plurality of protrusions or fins. In other embodiments, scraping mechanisms 222 can comprise a plurality of apertures or indentations configured to collect cells. However, any configurations of scraping mechanisms 222 are within the scope of the present invention.

In one embodiment, cell collector 202 is made of a medium density polyethylene materiel and has a length 352 of approximately one inch and a width 354 of approximately one-half inch. However, any other suitable materials and dimensions of cell collector 202 and handle 208 are within the scope of the present invention. For example, cell collector 202 can alternatively be made of nylon or cotton and have a diameter of greater than or less than one-half inch.

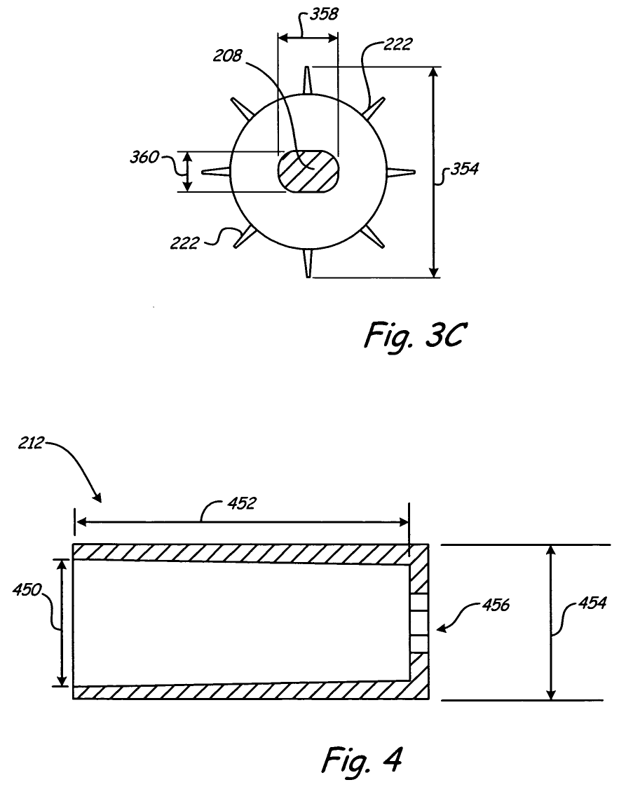

FIG. 4 is a side cross-sectional view of cup 212. Cup 212 has an exterior diameter 454 that is smaller than the interior diameter of tube 204 such that cup 212 can pass through tube 204. Further, interior width 450 and length 452 are large enough to accommodate a medical testing probe (i.e., cell collector 202 shown in FIG. 2). Aperture 456 is illustratively similar to aperture 218 shown in FIG. 2 and is configured to accommodate a handle.

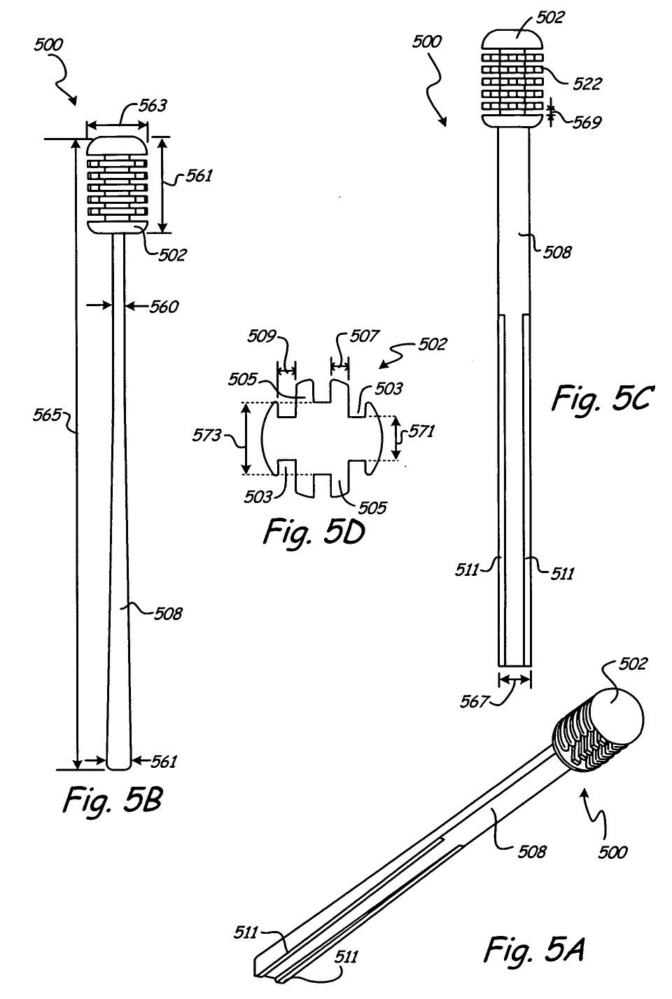

FIGS. 5A-5D illustrate a medical probe 500 in accordance with one embodiment of the present invention. Probe 500 can either be used as a stand-alone medical testing device (i.e., without a guide device or in conjunction with portions of device 200 disclosed in relation to FIG. 2). Cell collector 502 is attached to a distal end of handle 508 and is configured for cell retrieval. In accordance with one embodiment, cell collector 502 comprises a plurality of spaced flanges 522 configured to encourage cell collection. In one embodiment, flanges 522 are spaced 0.065 inches apart and have a thickness 569 of 0.065 inches. Further, in another embodiment, cell collector 502 has a diameter of approximately 0.625 inches and a height 561 of approximately one inch.

Additionally, handle 508 can include ridges 511. Ridges 511 create a taper in handle 508 (i.e., thickness 561 is larger than thickness 560). The taper encourages an enhanced gripping configuration and an increased flexibility in handle 508. In one embodiment, thickness 561 at the proximal end of handle 508 is approximately 0.25 inches and thickness 560 at the distal end of handle 508 is approximately 0.125 inches. In another example, the width 567 of handle 508 is approximately 0.320 inches. Further, in one embodiment, probe 500 has a length 565 of approximately 6.75 inches. In other embodiments, length 565 can be greater than or less than 6.75 inches.

FIG. 5D is an end cross-sectional view of cell collector 502. In accordance with one embodiment, cell collector 502 comprises a plurality of scraping mechanisms configured to encourage cell collection. As illustrated in FIG. 5D, in one embodiment, scraping mechanisms comprise a plurality of distinct raised protrusions 505 and indentations 503. In one example, protrusions 505 have a width 507 of 0.09 inches and indentations 503 have a width 509 of 0.09 inches. Further, cell collector 502 has interior widths 571 and 573 of 0.225 inches and 0.375 inches, respectively. However, any suitable configurations and dimensions of cell collector 502 and handle 508 are within the scope of the present invention.

One embodiment of the present invention pertains to a procedure for using medical probe 500. First, probe 500 is inserted into a vaginal cavity until cell collector 502 encounters resistance. In one example, probe 500 is inserted into it meets the opening of the cervix. Next, cell collector 502 is rotated (e.g., handle 508 is rotated clockwise three revolutions). Probe 500 is then withdrawn out of the vaginal cavity. It is noted that different depths of insertion are within the scope of the present invention.

As similarly described above with regard to FIG. 2, after removal of probe 500 (e.g., removal from a vaginal cavity), cell collector 502 and handle 508 can be placed inside a transport container containing a preserving solution. Alternatively, in some embodiments, handle 508 can be removed such that only cell collector 502 is placed in the transport container. The transport container can be mailed to a testing facility for medical diagnosis.

Although the present invention has been described with reference to preferred embodiments, workers skilled in the art will recognize that changes may be made in form and detail without departing from the spirit and scope of the invention.

What is claimed is:

1. A device for collecting biological samples from a cavity accessed through a vaginal orifice, wherein the device includes a probe, the probe comprising:
a proximal end;
a distal end; and
a cell collector located on the distal end and having a plurality of distinct raised protrusions.

2. The device of claim 1, wherein the probe is formed as one continuous piece.

3. The device of claim 1, wherein the plurality of distinct raised protrusions are dispersed so as to form at least one indention into the cell collector.

4. The device of claim 1, wherein the plurality of district raised protrusion comprises a plurality of elongated fins.

5. The device of claim 1 and further comprising a tubular guide mechanism shaped such that the probe can be extended through an interior of the guide mechanism.

6. The device of claim 5 and further comprising a cup mechanism shaped such that it can be slidingly inserted into the guide mechanism.

7. A system for collecting biological samples, comprising:
a probe having a proximal end and a distal end;
a cell collector located on the distal end of the probe; and
a preservation package configured to at least partially enclose the cell collector.

8. The system of claim 7 and further comprising a tubular guide mechanism shaped such that the probe can be extended through an interior of the guide mechanism.

9. The system of claim 8 and further comprising a cup mechanism configured to be slidingly inserted into the guide mechanism, the guide mechanism having an overall size and shape such that the cup mechanism remains inside the guide mechanism when the probe is extend out a distal end of the guide mechanism.

10. The system of claim 7, wherein the preservation package includes a preservation solution configured to encourage viability of a biological sample.

11. The system of claim 7 and further comprising instructions pertaining to tests to be performed on a biological sample, wherein the instructions include at least one of sexually transmitted diseases (STDs), cancer, and human papilloma virus(HPV).

12. A method for collecting a biological sample from a cavity accessed through a vaginal orifice, the method comprising:
inserting a cell collection probe into the orifice;
rotating the cell collection probe;
removing the cell collection probe from the orifice; and
placing the cell collection probe in a preservation tube.

13. The method of claim 12, wherein inserting the collection probe comprises:
inserting a tubular guide mechanism into the orifice, the guide mechanism being shaped such that the collection probe can be extended through an interior of the guide mechanism; and
extending the collection probe from a distal end of the guide mechanism.

14. The method of claim 13, wherein inserting the collection probe further comprises extending the collection probe from a cup mechanism within the guide mechanism.

15. The method of claim 12, wherein removing the cell collection probe from the orifice comprises:
retracting the cell collection probe into a tubular guide mechanism; and
removing the guide mechanism from the orifice.

16. The method of claim 15, wherein retracting the cell collection probe into the tubular guide mechanism comprises retracting into a cup mechanism within the guide mechanism.

17. The method of claim 16 and further comprising removing the cell collection probe from the guide mechanism.

18. The method of claim 17 and further comprising removing the cell collection probe and the cup mechanism from a proximal end of the guide mechanism.

19. The method of claim 12 wherein placing the cell collection probe in a preservation tube comprises placing the cell collection probe in a preservation solution and at least partially closing the preservation tube.

20. The method of claim 12 and further comprising indicating at least one test to be performed on the biological sample, wherein the at least one test is selected from a list comprising at least one of sexually transmitted diseases (STDs), cancer, and human papilloma virus(HPV).
